(19) United States
(12) Patent Application Publication
(10) Pub. No.: US 2007/0019161 A1
(43) Pub. Date: Jan. 25, 2007

(54) Eye measurement system providing for integration of elevation data and pachymetry data

(76) Inventor: Robert C. Clausing, Stansbury Park, UT (US)

Correspondence Address: Bausch & Lomb Incorporated, One Bausch & Lomb Place, Rochester, NY, 14604-2701, US

(21) Appl. No.: 11/186,590

(22) Filed: Jul. 21, 2005

(51) Int. Cl. A61B 3/10

(52) U.S. Cl. 351/212; 351/214

(57) ABSTRACT

An eye measurement system 10 includes a placido device 12 for obtaining elevation data of a patient's eye. A slit beam system 14 obtains pachymetry data of the eye. A computer processor 16 connected to the placido device 12 and the slit beam system 14 integrates the elevation data with the pachymetry data to obtain an accurate corneal topography model of the eye.

### BACKGROUND OF THE INVENTION

1. Field of the Invention

The present invention is related to eye measurement systems that provide a corneal topography model of an eye. More specifically, the present invention is directed to eye measurement systems that provide both a placido device and a slit beam system for measuring the eye.

2. Description of Related Art

Placido devices are widely known and have been around for many decades in the field of ophthalmology. A placido device generally consists of alternating light and dark rings which, when projected upon a patient's eye, provides curvature or elevation data of a person's eye. Placido devices however can take the form of other patterns, such as alternating light and dark patterns that can be described as spider-web, checkerboard, radial dots, and other patterns that may provide a practitioner with the curvature of a cornea.

Slit beam systems for providing pachymetry data of a cornea of an eye including anterior and posterior surfaces of a cornea are somewhat newer than a placido but are well known in the art. Patents such as U.S. Pat. Nos. 6,079,831; 5,735,283; and 5,512,966 all are examples of a slit beam system for providing a topographical map of an eye and have been incorporated in such commercial products as Bausch & Lomb Incorporated's OBSCAN II™ diagnostic instrument. All of the patents referred to in the previous sentence are commonly assigned to Bausch & Lomb Incorporated and are hereby incorporated by reference.

It is believed that most prior art slit beam measurement systems base the corneal model that is derived from the slit data on spherical theoretical models and, depending on the eye being measured, may be slightly different than the eye being measured. With the advent of a combined system using both a placido device and a slit beam measurement system from a common platform, it has been realized that the placido elevation data can be integrated with the slit beam pachymetry data to provide an accurate corneal model constructed from actual measurements of the cornea rather than a theoretical model.

Therefore, an advantage of the present invention is by using placido data in conjunction with the pachymetry data a corneal model is built from the actual measurements of the cornea rather than a theoretical model, which may or may not be an accurate representation of the cornea being measured.

### BRIEF DESCRIPTION OF THE DRAWING

FIG. 1 is a block diagram of an eye measurement system in accordance with the present invention;

FIG. 2 is a representation of a cornea with a slit beam being projected thereon;

FIG. 3 is a profile of a cornea along with a display of induced error in the measurement of the cornea;

FIG. 4 shows a cornea with a series of projected slits thereon and a meridian perpendicular to the slits; and

FIG. 5 is a three-dimensional representation showing projected slits aligned to lie on the meridian of FIG. 4, in accordance with the present invention.

### DETAILED DESCRIPTION OF THE PREFERRED EMBODIMENT

Slit scan technology such as that incorporated in Bausch & Lomb Incorporated's ORBSCAN II™ topography system consists of projecting a slit of light from a known source at a known angle onto a cornea. The above referenced patent applications describe this process in great detail. Through calibration and triangulation, the coordinates in three dimensions of the slit of light intersecting the cornea can be calculated. Error is introduced during calibration, through motor movement and eye movement. Each of these errors directly contributes to error in the calculation of the three-dimensional coordinates, as will be described below with reference to FIG. 2.

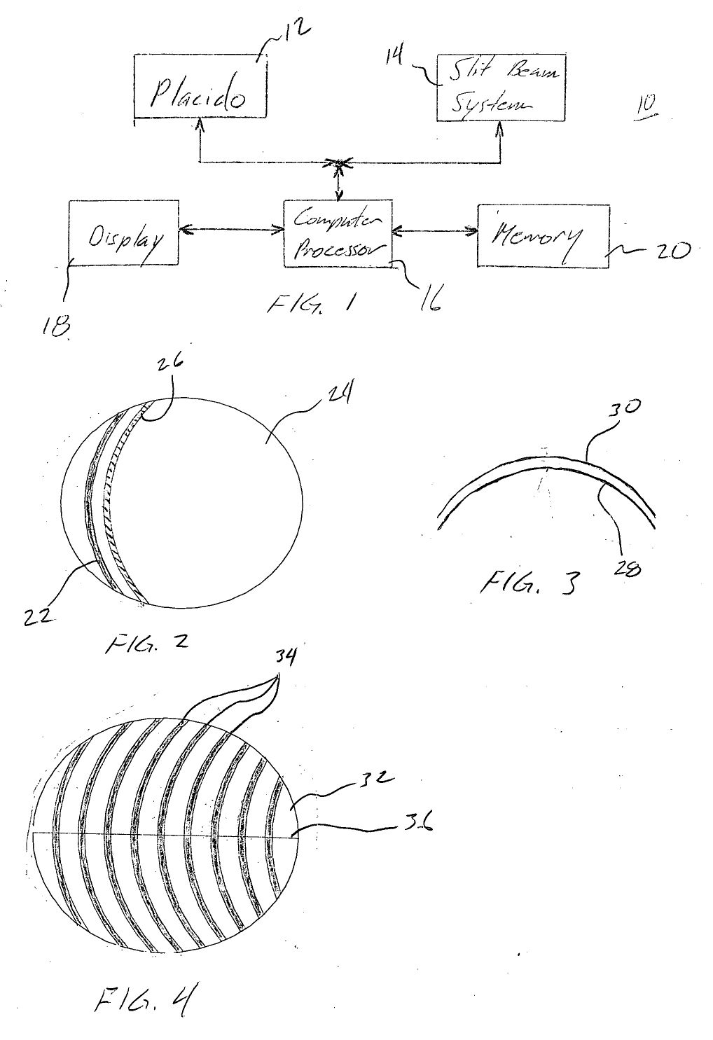

FIG. 1 shows an eye measurement system 10 that includes a placido device 12 for obtaining elevation data of a patient's eye in a known manner. System 10 also includes a slit beam system 14 for obtaining pachymetry data of the eye, such as known in the prior art and preferably includes Bausch & Lomb Incorporated's ORBSCAN II™ topography device or other similar instruments. System 10 further includes a computer processor 16 connected to the placido device 12 and the slit beam system 14. Computer processor 16 integrates the elevation data with a pachymetry data to obtain an accurate corneal topography model of the eye, as described in detail below. Also, preferably system 10 includes a display 18 for displaying the corneal topography model of the eye to a practitioner. System 10 further preferably includes a memory 20 connected to the computer processor 16 for storing the corneal topography model of the eye within system 10 for future reference and evaluation.

Referring now to FIG. 2, the errors in calculating the coordinates of the slit beam in three-dimensional space will always be present due to many factors, such as motor movement and eye movement and thus the errors can only be minimized. In order to construct a repeatable, accurate corneal model the errors must be compensated for, such as provided by the present invention.

Consider an individual slit 22 of FIG. 2 in two dimensions with respect to the actual surface of a cornea 24. The error in calculating the coordinates of the pachymetry data will cause the calculated pachymetry data to lie either above the actual surface of the cornea 24 or below the actual surface, depending on the direction of the error when the data is applied to the theoretical slit position in space. Therefore, the calculated slit position shown at 26 will actually be somewhat misplaced relative to the actual projected coordinates of the slit 22.

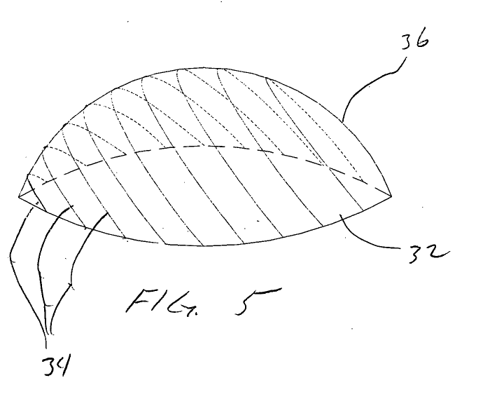

By sampling the placido data for surface normals, in a known manner, along a meridian perpendicular to the projected slits on the cornea, a curve can be constructed to accurately represent the surface profile along the sample meridian. Such a sample meridian profile can be seen in FIG. 3 at 28 and a calculated pachymetry data from the slit beam system may be as shown at 30. Because a human cornea is very close to spherical, all the peaks of each slit data set should intersect with the surface profile created from the meridian perpendicular to the slits, as shown in FIG. 4. FIG. 4 shows a cornea 32 with a series of projected slits 34 with a perpendicular meridian 36.

Adjusting the overall elevation of each individual slit, such that a peak of each slit along a perpendicular meridian of a set of slits intersects the surface profile created from the placido data aligns each slit in three-dimensional space, with respect to the other slits. As shown in FIG. 5, a cornea 32 with slits 34 aligned along perpendicular meridian 36 adjusted according to placido data causes the pachymetry data from the slits to be more precisely and accurately aligned according to the particular cornea 32 being measured. The adjustment of the individual slits to align with the meridian calculated from the placido data may be accomplished by any number of known curve-fitting software and mathematical models. In this way, the computer processor 16 integrates the elevation data and the pachymetry data such that a peak of the pachymetry data intersects with the surface profile created from the elevation data.

The system 10, by shifting the posterior pachymetry data from the slit beam system 14 in the same manner as the anterior corneal data was shifted to match the placido data, yields a highly accurate three-dimensional mathematical and graphical representation of the entire cornea. The present invention leverages the well proven and accepted accuracy of the placido device to create a very accurate representation of both the anterior and posterior corneal surfaces.

I claim:

1. An eye measurement system comprising:
a placido device for obtaining elevation data of a patient's eye;
a slit beam system for obtaining pachymetry data of the eye; and
a computer processor connected to the placido device and the slit beam system for integrating the elevation data with the pachymetry data to obtain an accurate corneal topography of the eye.

2. The invention of claim 1, wherein the computer processor integrates the elevation data and the pachymetry data, such that a peak of the pachymetry data intersects with a surface profile created from the elevation data.

3. The invention of claim 2, wherein the computer processor further shifts a posterior pachymetry data set obtained from the slit beam system to obtain an accurate three-dimensional representation of a posterior corneal surface of the eye.

4. The invention of claim 1 further including a display connected to the computer processor for displaying the corneal topography model of the eye.

5. The invention of claim 1 further including memory connected to the computer processor for storing the corneal topography model of the eye.

6. The invention of claim 2, wherein the peak of the pachymetry data is aligned to lie along a meridian perpendicular to the pachymetry data and calculated from the elevation data.

7. A method of obtaining a corneal topography model of an eye, comprising the steps of:
obtaining elevation data of the eye with a placido device;
obtaining corneal pachymetry data of the eye with a slit beam system; and
integrating, with a computer processor, the elevation data and the corneal pachymetry data to obtain an accurate corneal topography model of the eye.

8. The method of claim 7, wherein the integrating step includes adjusting a peak of the pachymetry data to intersect with a surface profile created from the elevation data.

9. The method of claim 8, further including the step of shifting a posterior pachymetry data set obtained from the slit beam system to obtain an accurate three-dimensional representation of a posterior corneal surface of the eye.

10. The method of claim 7 further including displaying the corneal topography model of the eye on a display.

11. The method of claim 7 further including storing, in a memory, the corneal topography model of the eye.
